(19) United States
(12) Patent Application Publication
(10) Pub. No.: US 2024/0009395 A1
(43) Pub. Date: Jan. 11, 2024

(54) BLOW-FILL-SEAL (BFS) ASSISTED VIAL DISPENSING SYSTEMS AND METHODS

(71) Applicant: Koska Family Limited, East Sussex (GB)

(72) Inventors: Marc Koska, Dartmouth (GB); Max Hannon, Beverley (GB)

(21) Appl. No.: 18/371,429

(22) Filed: Sep. 21, 2023

Related U.S. Application Data
(63) Continuation of application No. PCT/IB2022/053130, filed on Apr. 4, 2022.
(60) Provisional application No. 63/170,412, filed on Apr. 2, 2021.

(51) Int. Cl. A61M 5/24

(52) U.S. Cl. CPC A61M 5/2425; A61M 2205/581; A61M 2205/582; A61M 2205/43

(57) ABSTRACT

Systems and methods for dispensing Blow-Fill-Seal (BFS) vials such as pre-filled vials containing a vaccine or other medicament via utilization of an assistive dispensing device to selectively compress the fluid reservoir of the BFS vial is shown and described. The assistive dispensing device includes one or more protrusions to aid in applying compressive force to assist in dispensing the liquid from the vial, and may also include a first dosage setting member and a second dosage setting member, that engage each other to limit movement of the one or more protrusions when the proper compression force has been reached.

### CROSS-REFERENCE TO RELATED APPLICATIONS

This application is a Continuation Application of PCT Application No. PCT/162022/053130, which PCT Application claims benefit and priority under 35 U.S.C. § 119(e) to, and is a Non-provisional of, U.S. Provisional Patent Application No. 63/170,412 filed on Apr. 2, 2021 and titled “BLOW-FILL-SEAL (BFS) VIAL DISPENSING SYSTEMS AND METHODS.” Each of these Applications is hereby incorporated by reference herein in its entirety and for all purposes.

### TECHNICAL FIELD

The present disclosure describes systems, methods and devices for assisted dispensing of Blow-Fill-Seal (BFS) pre-filled, single-dose medicament delivery systems. In accordance with some embodiments, an assisted dispensing device may be embodied as an adjustable accessory component (referred to in some embodiments as a “cover”) that may be utilized with a BFS medicament delivery system and function to assist with gripping of a BFS vial component of the BFS medicament delivery system and/or dispensing of the contents thereof.

### SUMMARY OF VARIOUS EMBODIMENTS

Embodiments of the disclosed subject matter provide systems, assemblies, kits, devices and methods for assisting with the medical delivery of a fluid agent to a patient. Using a blow-fill-seal (BFS) fabrication technique, a BFS component (such as a vial) is pre-filled with a fluid (e.g., liquid) agent intended for administration to a patient. In some embodiments, the liquid agent is administered via a needle or cannula coupled to the BFS vial (e.g., via use of a connector component).

The BFS medical delivery systems developed by Applicant provides a cost-effective option for pre-filled, single-dose delivery of fluid agents (e.g., vaccines or medicaments). In some embodiments, the BFS vials comprising components of such medical delivery systems are pressure activated by a user gripping and compressing a squeezable reservoir to dispense the fluid agent. Example dosage sizes for many fluid agents dispensed through Applicant's BFS medical delivery systems can be quite small (e.g., between three-tenths of a milliliter (0.3-ml) and three milliliter (3.0-ml)) and the geometry of BFS vials containing such substances are accordingly small in size. Applicant has recognized that while BFS medical delivery systems described herein provide a low-cost, sterile, single-use injection, the small geometry size of the BFS vials compared to the human hand may prove challenging for some users during dispensing. For example, users with larger fingers, low grip strength, or mobility issues (e.g., arthritis) may find it difficult to readily achieve a sufficient grip on a small, pressure-activated BFS delivery system (or a portion thereof such as the squeezable reservoir). This may result in unsatisfactorily compressing of the BFS chamber or reservoir, and difficulty in dispensing the proper pre-filled dose as a result.

Embodiments described herein provide systems, methods, kits and/or devices for assisted dispensing of BFS vials by providing an accessory device that may comprise an adjustable cover operable to aid with gripping and dispensing, e.g., that acts as a lever to aid with gripping and proper dispensing by amplifying the pressure exerted by the user, and can also provide proper dosing by limiting movement of the dispensing collar.

### BRIEF DESCRIPTION OF THE DRAWINGS

The figures depict embodiments for purposes of illustration only. The accompanying figures are not necessarily drawn to scale, emphasis instead being placed upon illustrating the principles disclosed herein. The figures are included to further the understanding of the various aspects and embodiments and are incorporated in and constitute a part of this specification, but are not intended as a definition of the limits of any particular embodiment. In the figures, each identical or nearly identical component that is illustrated in various figures is represented by a like numeral. For example, the same or similar elements throughout various embodiment may include the same two base reference numbers, preceded with a different numeral. For purposes of clarity, not every component may be labeled in every figure. One skilled in the art will readily recognize from the following description that alternative embodiments of the systems and methods illustrated herein may be employed without departing from the principles described herein, wherein:

FIG. 1A is a perspective view of a BFS injection system employing an assisted dispensing collar with a vial, according to one or more embodiments of the disclosed subject matter;

FIG. 1B is an exploded view of the BFS injection system and collar of FIG. 1A illustrating attachment of the assisted dispensing collar;

FIG. 1C is an exploded view of the BFS injection system and collar of FIG. 1A illustrating attachment of the assisted dispensing collar with an alternate vial;

FIG. 1D is a perspective view of the BFS injection system and collar of FIG. 1C;

FIG. 1E is a perspective view of the BFS injection system of FIG. 1A illustrating attachment of the dispensing collar to a BFS vial and injection assembly;

FIG. 1F is a perspective view of the BFS injection system of FIG. 1A a first, attached position;

FIG. 2A is perspective view of the assisted dispensing collar of FIG. 1A;

FIG. 2B is a top view of the dispensing collar of FIG. 2A;

FIG. 2C is a front view of the dispensing collar of FIG. 2A;

FIG. 3A is a front view of the dispensing collar and a front, cross-sectional view of the BFS vial and injection assembly of FIG. 1F in the first, attached position;

FIG. 3B illustrates the BFS injection system of FIG. 3A in a second, compressed position;

FIG. 3C illustrates the BFS injection system of FIG. 3A in a third, injecting position;

FIGS. 4A and 4B are perspective and side views of a BFS vial for use with the BFS injection system according to one or more embodiments of the disclosed subject matter;

FIGS. 4C and 4D are perspective and side views of a BFS vial for use with the BFS injection system according to one or more embodiments of the disclosed subject matter;

FIG. 5A is a perspective view of a BFS injection system employing an assisted dispensing collar with a vial, according to one or more embodiments of the disclosed subject matter;

FIG. 5B is an exploded view of the BFS injection system and collar of FIG. 5A illustrating attachment of the assisted dispensing collar;

FIG. 5C is an exploded view of the BFS injection system and collar of FIG. 5A illustrating attachment of the assisted dispensing collar with an alternate vial;

FIG. 5D is a perspective view of the BFS injection system of FIG. 5B a first, attached position;

FIG. 5E is a perspective view of the BFS injection system of FIG. 5C a first, attached position;

FIG. 6A is perspective view of the assisted dispensing collar of FIG. 5A;

FIG. 6B is a front view of the assisted dispensing collar of FIG. 6A;

FIG. 6C; is a top view of the dispensing collar of FIG. 6A;

FIG. 6D; is a bottom view of the dispensing collar of FIG. 6A;

FIG. 7A is a front view of the dispensing collar and a front, cross-sectional view of the BFS vial and injection assembly of FIG. 5D in the first, attached position;

FIG. 7B illustrates the BFS injection system of FIG. 7A in a second, compressed position;

FIG. 7C illustrates the BFS injection system of FIG. 7A in a third, injecting position;

FIG. 8A is a perspective view of a BFS injection system employing an assisted dosing and dispensing collar, according to one or more embodiments of the disclosed subject matter;

FIG. 8B is a perspective view of the BFS injection system of FIG. 8A illustrating attachment of the dosing and dispensing collar to a BFS vial and injection assembly;

FIG. 9A is a perspective view of the dosing and dispensing collar of FIG. 8A in a flat, installation configuration;

FIG. 9B is a perspective view of the dosing and dispensing collar of FIG. 9A in an initial folded configuration;

FIG. 9C is a front view of the dosing and dispensing collar of FIG. 9A;

FIG. 9D is a bottom view of the dosing and dispensing collar of FIG. 9A;

FIG. 9E is a side view of the dosing and dispensing collar of FIG. 9A;

FIG. 10A is a cross-sectional view of the BFS injection system including the dosing and dispensing collar of FIG. 8A in a first, open and attached position;

FIGS. 10B and 10C illustrate the arms of the BFS injection system of FIG. 10A moving into engagement with a BFS vial according to one or more embodiments of the disclosed subject matter;

FIG. 10D is a perspective view of the BFS injection system of 8A in a second, engaged or compressed position with the BFS vial; and

FIGS. 10E-10G are perspective views illustrating various sequential stages in the use of the BFS injection system of FIG. 1 to inject a dose from the BFS vial to a target (e.g., patient).

### DETAILED DESCRIPTION

### I. Introduction

Embodiments of the present disclosure provide systems and methods for assisted dispensing of pre-filled medical delivery assemblies, for example, medicaments from a BFS dispensing system. An assisted vial dispensing device is disclosed that aids a user in squeezing a body of the BFS dispensing system, for example a fluid chamber or collapsible fluid reservoir, to dispense the medicaments. The assisted dispensing device may comprise, in some embodiments, a pair of arms for gripping by the user that provide leverage to assist in pressure-activating, i.e., squeezing, the BFS vial during injection to dispense the proper pre-filled dose. Alternatively or additionally, it may be desirable in some circumstances to customize the compression delivered to a common-sized BFS vial, for example, to use the same style BFS vials to deliver different doses based on the amount of compression (e.g., to deliver a full dose of medicaments to an adult versus delivering a half dose of the medicaments to a child). Embodiments of the disclosed subject matter may address one or more of the above-noted desirable results and/or, among other things.

### II. BFS Assisted Dispensing Systems and Methods

The examples of the systems and methods discussed herein are not limited in application to the details of construction and the arrangement of components set forth in the following description or illustrated in the accompanying drawings. It will be understood to one of skill in the art that the apparatus is capable of implementation in other embodiments and of being practiced or carried out in various ways. Examples of specific embodiments are provided herein for illustrative purposes only and are not intended to be limiting. Also, the phraseology and terminology used herein is for the purpose of description and should not be regarded as limiting. Any references to examples, embodiments, components, elements or acts of the apparatus herein referred to in the singular may also embrace embodiments including a plurality, and any references in plural to any embodiment, component, element or act herein may also embrace embodiments including only a singularity (or unitary structure). Likewise, components disclosed as a single element may be made from separate elements or devices. References in the singular or plural form are not intended to limit the presently disclosed apparatus, its components, acts, or elements. The use herein of “including,” “comprising,” “having,” “containing,” “involving,” and variations thereof is meant to encompass the items listed thereafter and equivalents thereof as well as additional items.

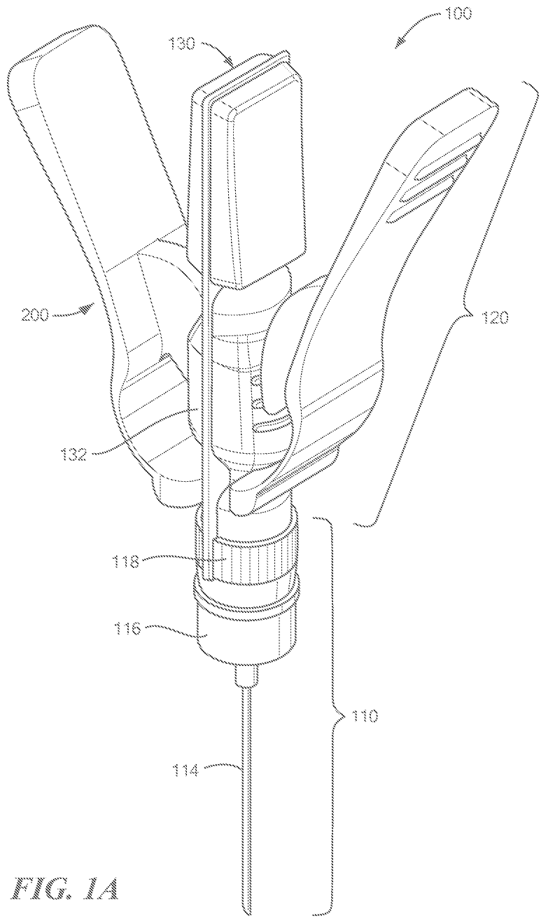

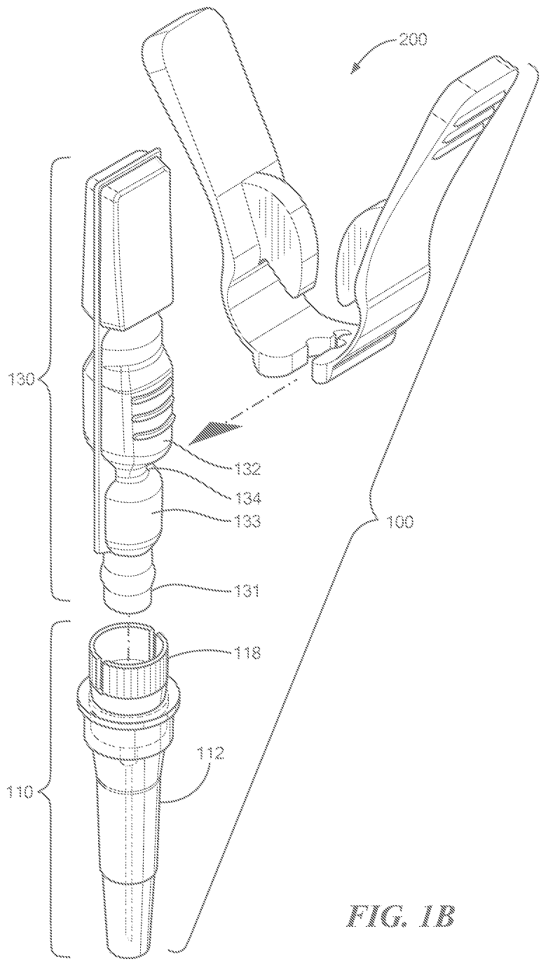

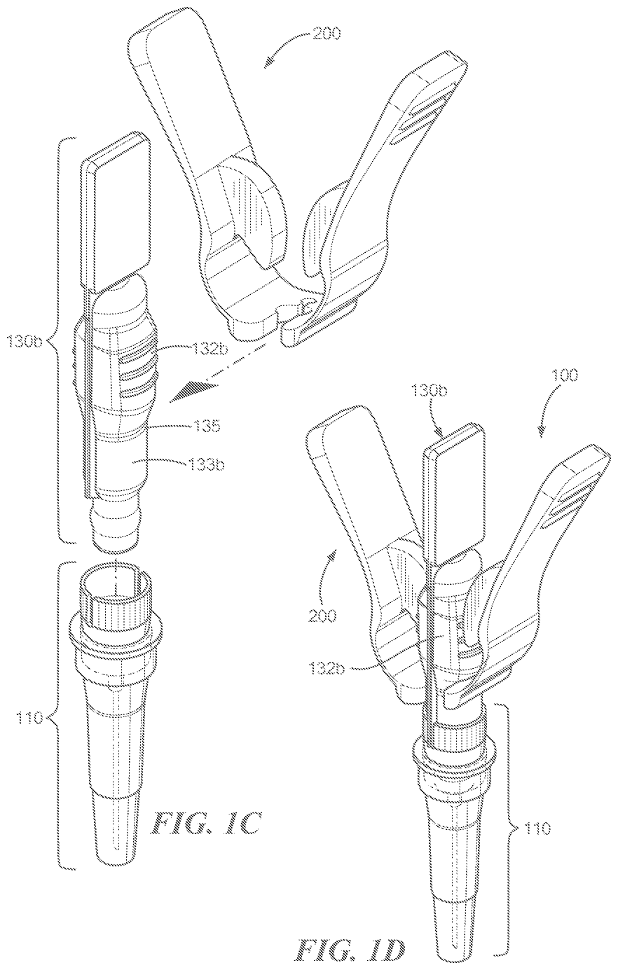

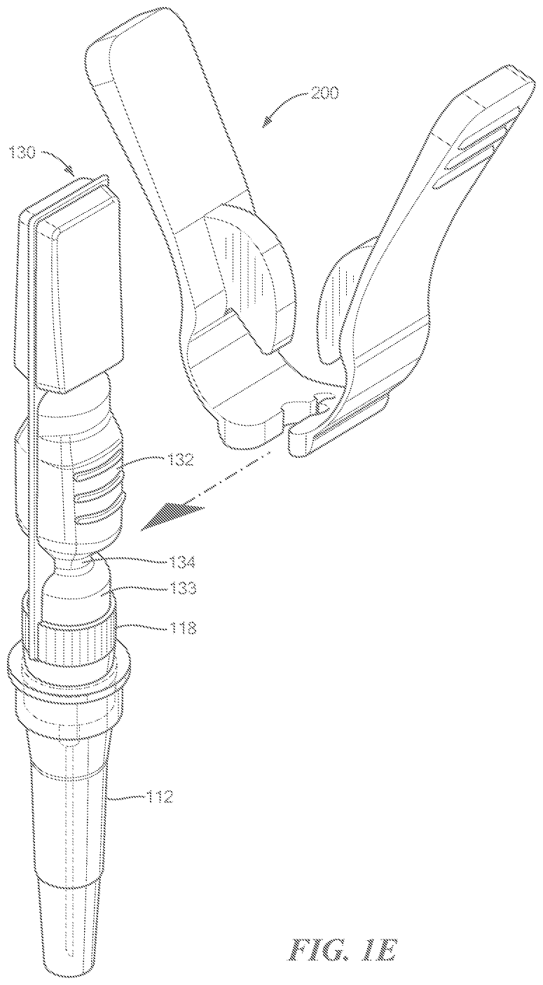

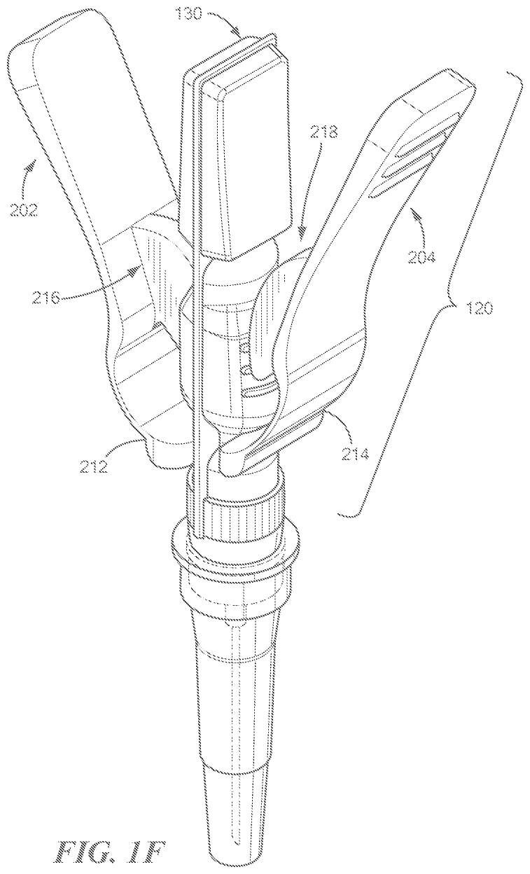

Referring initially to FIGS. 1A-1E there is illustrated an exemplary BFS assisted dispensing system 120 comprising BFS vial 130 and an assisted dispensing device, for example a lever and/or collar 200, for actuating at least one fluid reservoir 132 of the BFS vial 130 (also referred to as a bottle or container). The fluid reservoir 132 may be filled with a fluid, for example air, and be fluidly connected to a dispensing chamber 133 that stores houses, and/or is to be filled with one or more medicaments and/or desirable fluid substances, such as saline solution, vaccine, insulin, and/or other medical substances. In some embodiments, the fluid reservoir 132 may additionally have other fluids stored therein. The assisted dispensing collar 200 can be releasably secured to the BFS vial 130 (FIG. 1F), and may be used to aid in forcing the fluid from the upper fluid reservoir 132 in order to deliver the medicaments from the lower dispensing chamber 133.

Assisted collar 200 can be used to apply a compression force to the fluid reservoir 132 in a direction that may be substantially perpendicular thereto, in order to cause the contents therein (e.g., all or some of the substances stored, housed, or filled within the reservoir 132) to be dispelled from the fluid reservoir. In order to deliver the fluid from the dispensing chamber 133 to the ultimate target (i.e., patient), the BFS vial 130 can be releasably coupled to an injection assembly 110. The BFS assisted dispensing system 120 and injection assembly 110 together form the BFS injection system 100. The injection assembly 110 may include a needle 114 extending from a needle hub 116, a connector 118, and a releasable cap 112 can be provided to protect the exposed end of the needle until use.

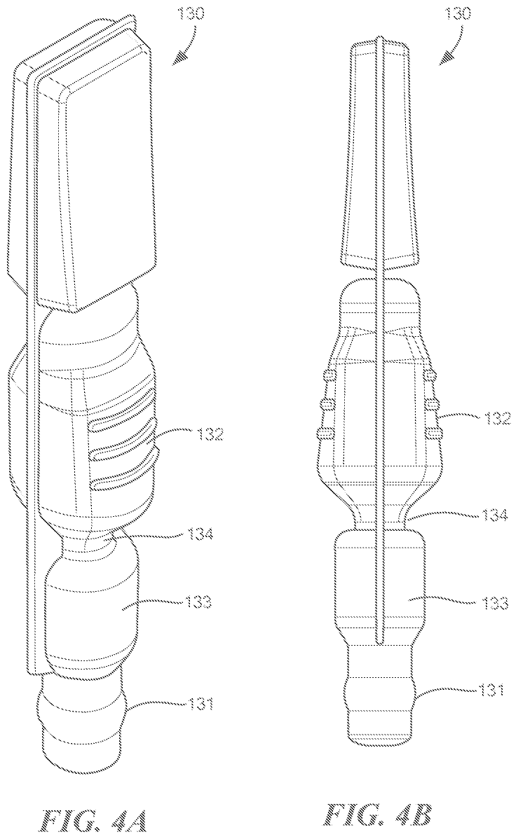

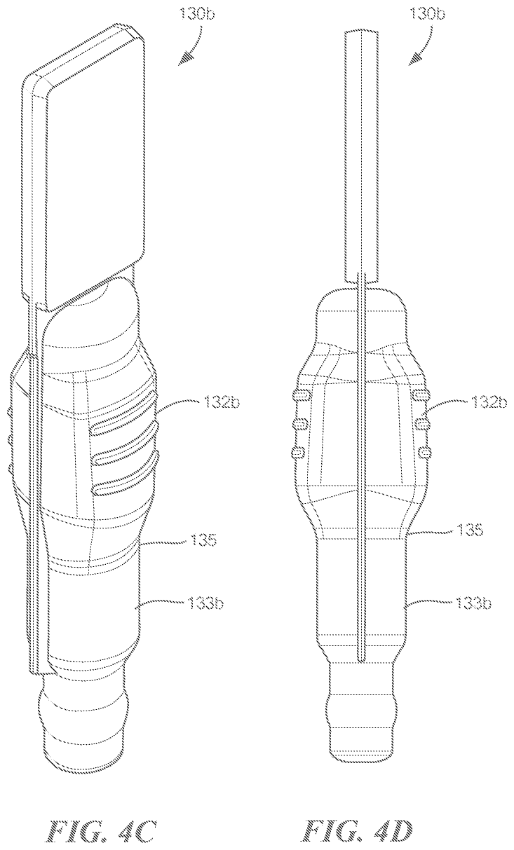

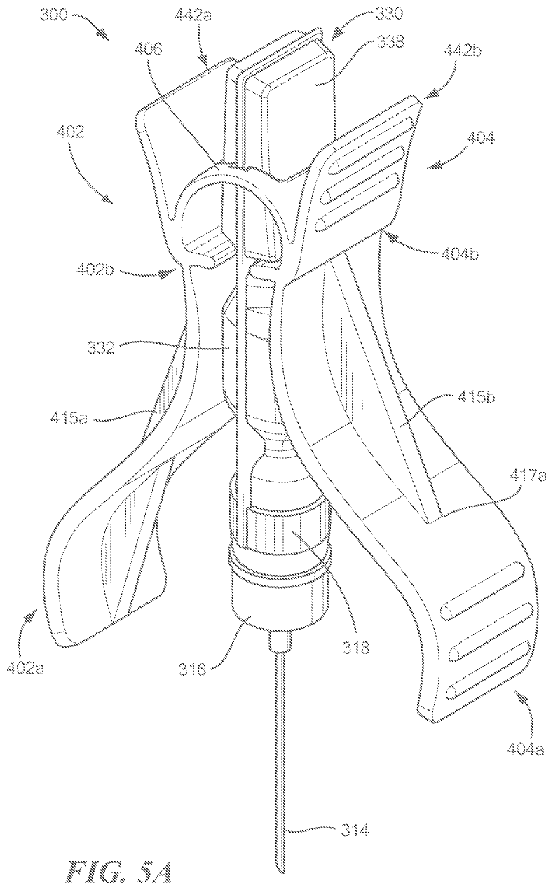

The assisted collar 200 can be secured, supported mounted on and/or coupled to the BFS vial 130 (e.g., as depicted in FIG. 4A and/or FIG. 4B), for example, by sliding on or securing to an inwardly tapered connection portion 134 (also referred to as a neck), which fluidly connects the upper fluid reservoir 132 to the lower dispensing chamber 133. Alternately, as illustrated in FIGS. 1C, 4C and 4D, a more unitary vial 130b including an upper fluid reservoir 132b and lower dispensing chamber 133b may be provided with a non-inwardly tapered connection, and instead includes a complimentary connection portion 135. BFS vial 130b may house one or more fluids including, for example, air and/or liquids. In some embodiments, the dispensing collar 200 is releasably secured to the BFS vial 130 and may be designed to be removed therefrom after use and re-used with another BFS vial.

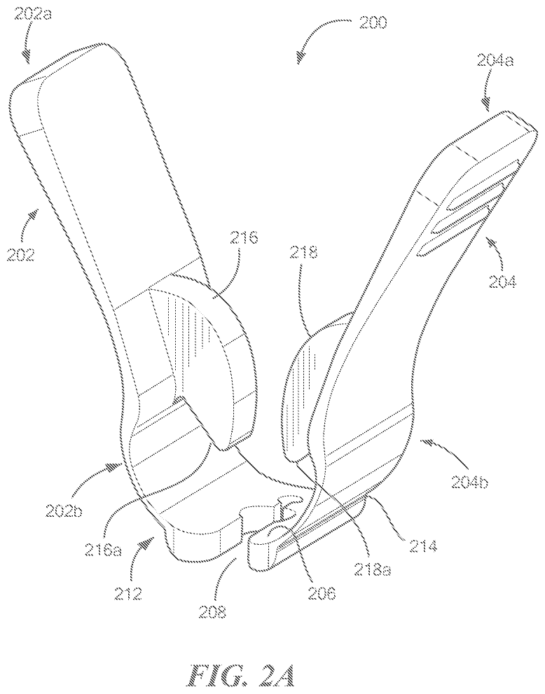

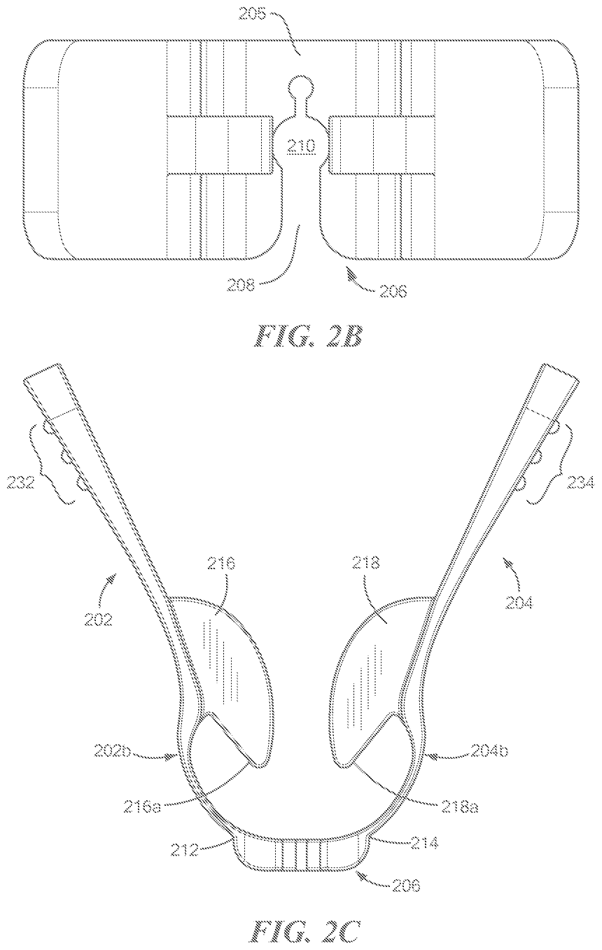

Referring now to FIGS. 2A-2C, an exemplary dispensing collar 200 is illustrated that may include a first arm 202 having a gripping end 202a and a pivoting end 202b, a second arm 204 having a gripping end 204a and a pivoting end 204b, and an attachment member 206 disposed therebetween. The first arm 202 may be connected to the attachment member 206 via a first pivot portion 212. On an opposite side of the attachment member 206, the second arm 204 can be connected thereto via a second pivot portion 214. Each pivot portion 212, 214 defines a flexible joint by which the first and second arms 202, 204 can be rotated toward each other for actuating the BFS vial 130. In the exemplary embodiment, the first arm 202 includes at least one first protrusion 216 supported on an inner surface thereof, above the first pivot portion 212. The protrusion 216 may be arcuate in shape and include an extension 216a. Likewise, the second arm 204 supports at least a second protrusion 218 disposed at an opposing location to protrusion 216, and is similar in size and shape, and may also include an arcuate shape and an extension 218a. However, in some embodiments, other sizes, shapes, numbers or arrangements of protrusions are also possible. It should also be clear that in some embodiments, the assisted collar 200 may be provided and/or may exist in the absence of the BFS vial 130, and vice versa.

Arms 202, 204, attachment member 206, protrusions 216, 218, and pivot portions 212, 214 can be made as a single, unitary structure as illustrated, or may be made as separate components joined together. For example, the first pivot portion 212, the second pivot portion 214, or both can be formed as a living hinge or other sufficiently flexible or bendable structure. A grip portion 232 and 234 may be provided on an outer surface of the first arm 202 and the second arm 204, respectively. In some embodiments, the grip portions 232, 234 may be knurled or other structural features designed to enhance gripping by a user. A user of the BFS assisted dispensing system 120 can grasp the grip portions 232, 234 (e.g., using a thumb and index finger) to provide the desired compressive force for actuation of the first and second arms 202, 204.

In the illustrated example, attachment member 206 has an opening or connector slot 210 that is sized and shaped to substantially mate with the connection portion of the BFS vial 130. As best shown in FIG. 2B, access to the connector slot 210 can be provided via an access channel 208, which may be tapered and has a width that is less than a corresponding width of the BFS vial connection portion 134 or 135, depending on the style vial. For example, the body 205 of the attachment member 206 defining the access channel 208 can be made of a material that deflects outward as the BFS vial connection portion is inserted therein, in order to increase the width of the access channel 208. Once the connection portion is received through channel 208 and into connector slot 210, the body 205 of the attachment member 206 can return back toward its original configuration, thereby releasably retaining the dispensing collar 200 to the BFS vial 130 as shown in FIG. 1A.

By connecting the dispensing collar 200 below the vial 130/130b at a location below reservoir 132/132 with the arms 202, 204 extending upward and outward therefrom, a point of leverage is provided for a user that is at the proximal end of the BFS assistive dispensing system 120, opposite the injection or distal end. The assisted dispensing collar 200 acts as a fulcrum when the user grips the arms 202, 204 and applies pressure to squeeze the arms 202, 204 toward each other. In this manner, the dispensing collar 200 assists in the manual actuation of the BFS injection system to dispense and/or inject contents from the fluid reservoir 132 of the BFS vial 130 by amplifying or increasing the pressure produced by the user.

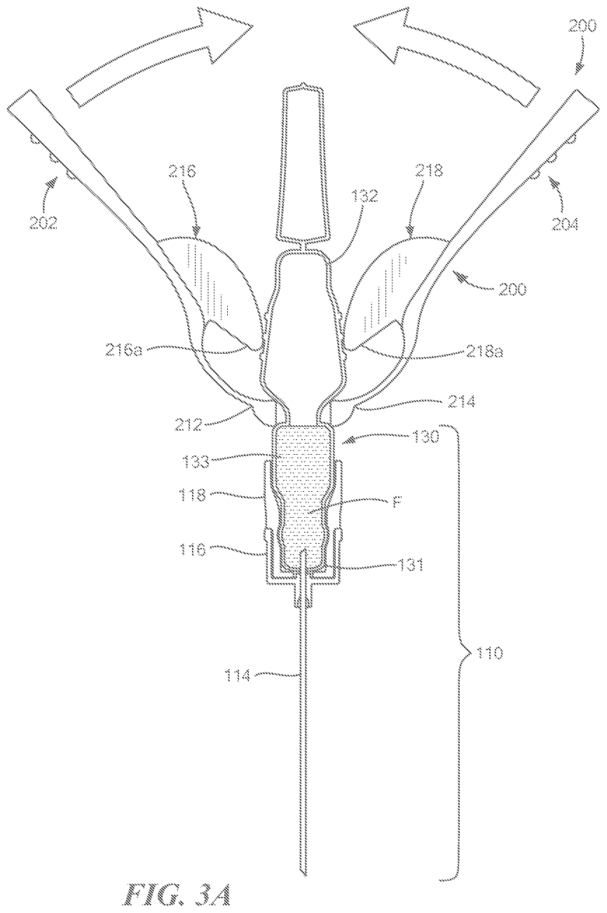

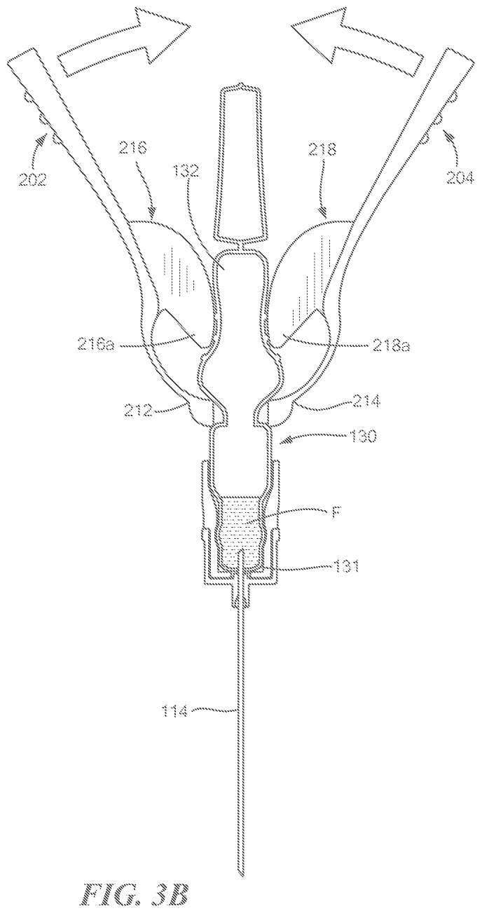

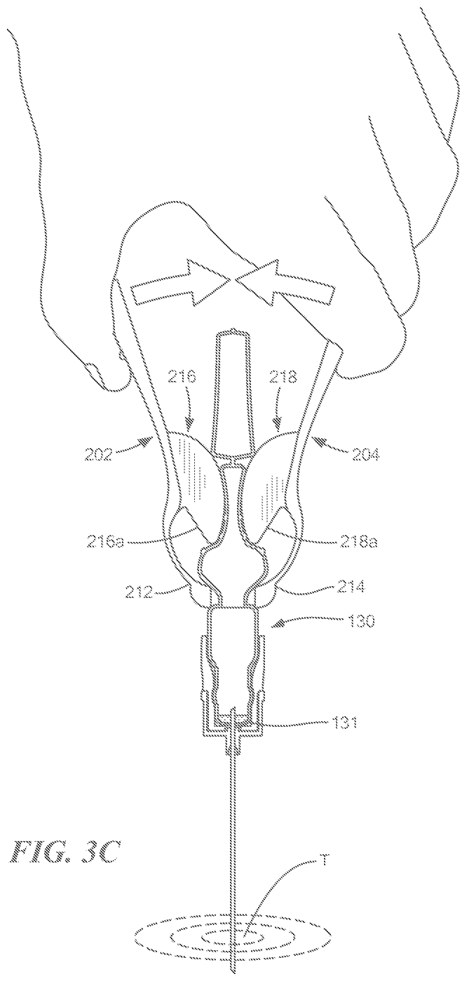

Referring now to FIGS. 3A-3C, connector 118 of the injection assembly 110 may fluidically connect the needle hub 116, and the needle 114 to an outlet end 131 of the BFS vial 130. In some exemplary embodiments, the dispensing chamber 133 is in fluid communication with fluid reservoir 132 via connection portion 134 and with the proximal end of needle 114, so that the fluid from reservoir 132 enters the chamber 133 forcing fluid in the chamber 133 to be dispensed through the needle 114 for injection. Once inserted onto the connection portion 134, the extensions 216a, 218a are located adjacent to the fluid reservoir 132 of the BFS vial 130, for example, in contact therewith (FIG. 3A), without applying a compressive force thereto. This is the first, attached position. Further movement of the first and second arms 202, 204 toward each other, as shown by the arrows of FIG. 3B, causes the extensions 216a, 218a to push on the external walls of the fluid reservoir 132, and the arcuate surface to ride against the surface of the fluid reservoir 132, thereby applying a compressive force that deforms the fluid reservoir to cause expulsion (e.g., all or some) of the air or other fluid from the reservoir 132. This is the second, compressed position. Continued pressure applied by the user causes further movement of the protrusions 216, 218 toward each other as shown in FIG. 3C into the third, injecting position. In this position, the continued pressure applied by the user through the assisted collar 200 forces the air out of the fluid reservoir 132 and into and/or through dispensing chamber 133, and the liquid from the dispensing chamber 133 through the needle 114 and into the target injection site “T”. This occurs without the user having to apply the compressive force that would be needed absent the assisted collar, by virtue of the leverage provided through the assisted dispensing collar. Once injected, the assistive collar 200 may be removed and re-used and the empty vial 130 and injection assembly 110 discarded.

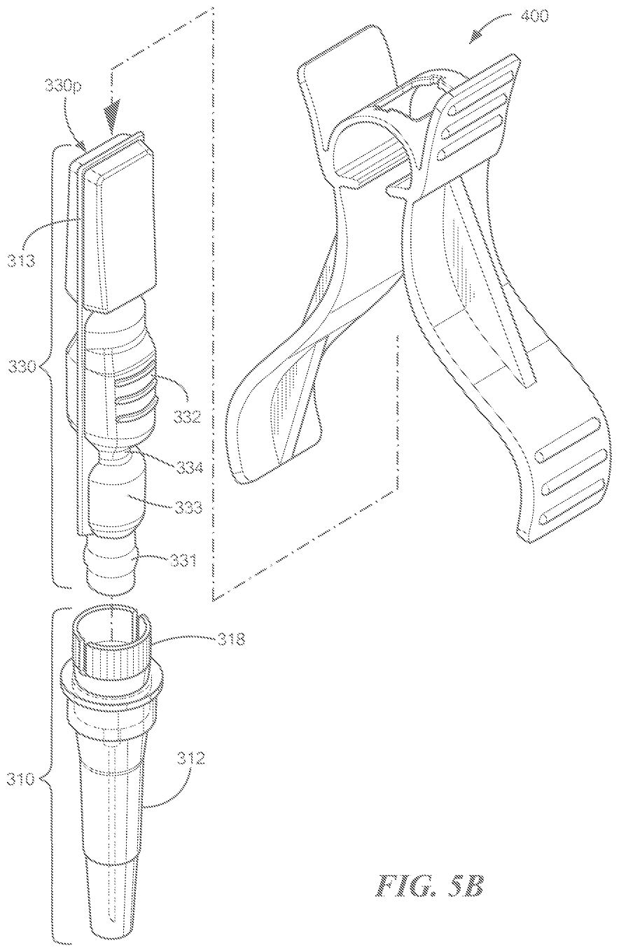

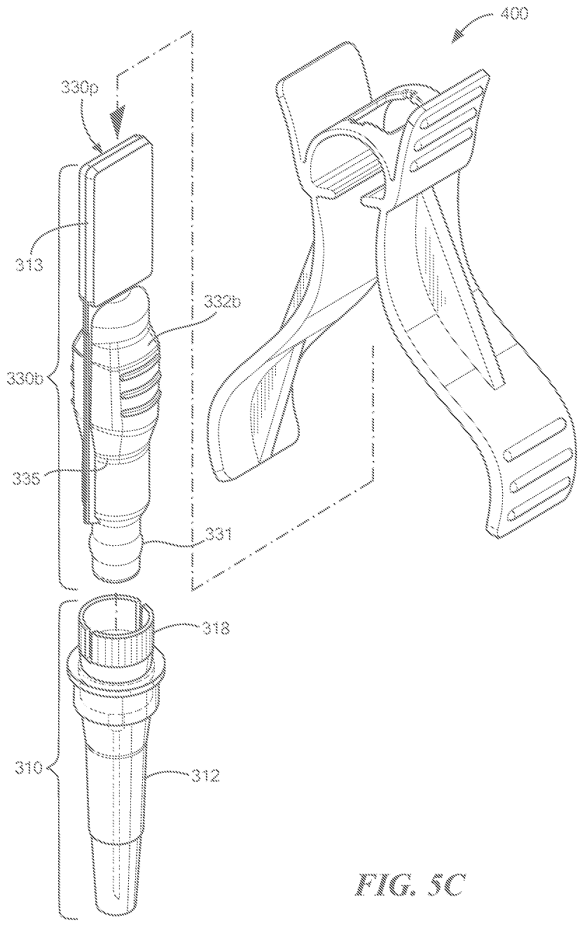

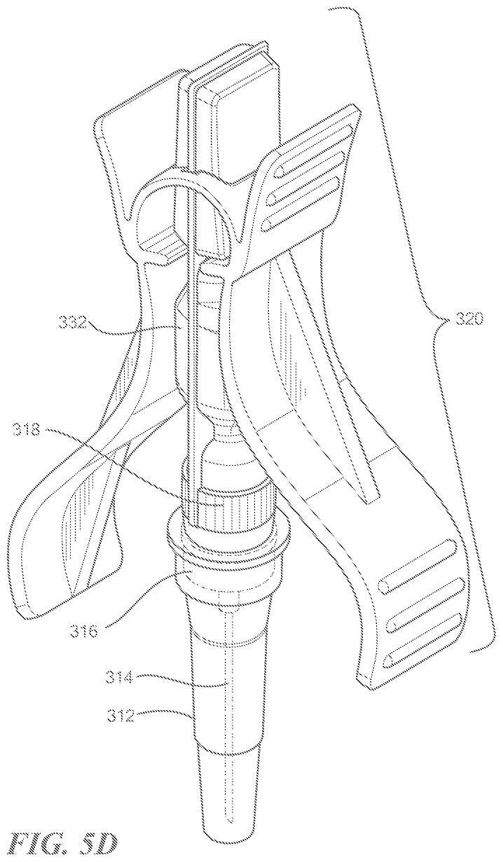

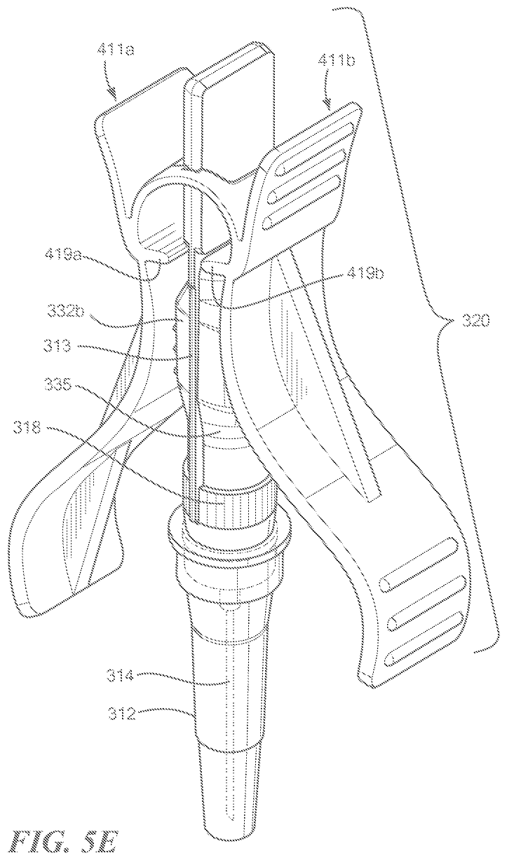

Referring not to FIGS. 5A-7C, a second exemplary embodiment of a BFS injection system 300 including a BFS assisted dispensing system 320 that employs an assisted dispensing device, for example a lever and/or collar 400 for actuating at least one fluid reservoir 332 (e.g., having a constriction 334) of an exemplary BFS vial 330 is illustrated (or an alternate design featuring a unitary fluid reservoir 332b, 335). The assistive dispensing collar 400 is similar in function to the assistive dispensing collar 200, except that the connection portion for the assisted dispending collar 400 is at a different location, and the point of leverage is provided adjacent the distal end of the BFS dispensing system 320. The assisted dispensing collar 400 still acts as a fulcrum when the user grips the arms 402, 404 and applies pressure to squeeze the arms 402, 404 toward each other, but the user grips the assistive dispensing collar 400 adjacent the injection assembly 310 in this exemplary embodiment.

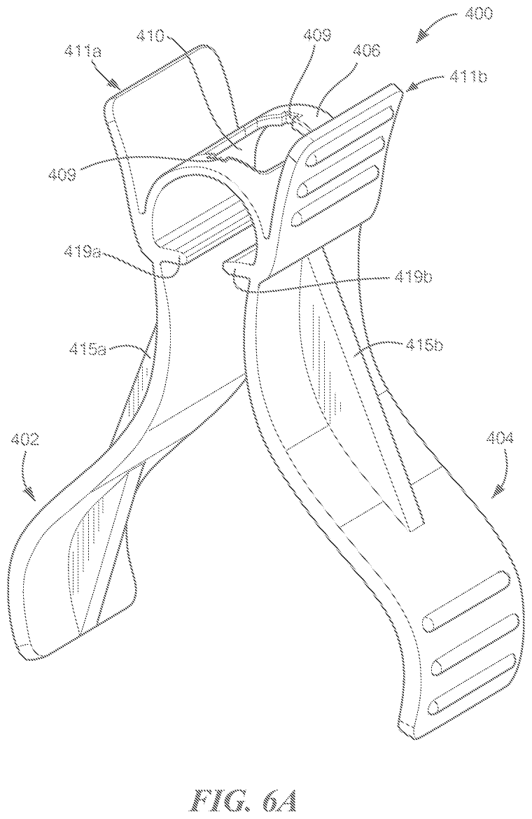

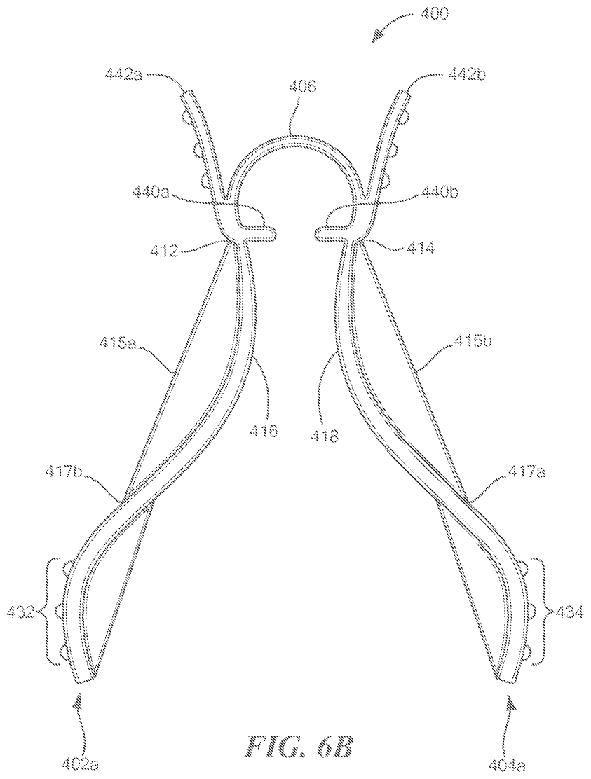

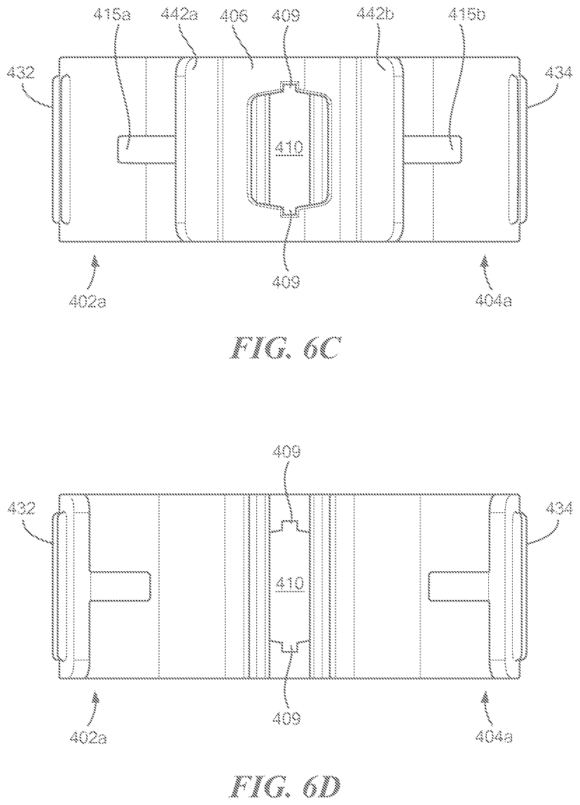

Referring now to FIGS. 6A-6D, the exemplary dispensing collar 400 is illustrated that may include a first arm 402 having a gripping end 402a and a pivoting end 402b, a second arm 404 having a gripping end 404a and a pivoting end 404b, and an attachment member 406 disposed therebetween, as described hereinabove. The first arm 402 may be connected to the attachment member 406 via a first pivot portion 412. On an opposite side of the attachment member 406, the second arm 404 can be connected to the attachment member 406 via a second pivot portion 414. Each pivot portion 412, 414 defines a flexible joint by which the first and second arms 402, 404 can be rotated toward each other for actuating the BFS vial 330.

The assisted dispensing collar 400 can be releasably secured to the BFS vial 330 by engagement of interior mating flanges 440a-b with one or more depressions or joints of the BDS vial 330 (e.g., such mating being facilitated by application of inward radial pressure to reverse fulcrum tabs/portions 442a-b), and may be actuated to aid in applying a compressive force to the fluid reservoir 332. The compressive force applied by the assisted dispensing collar 400 causes the contents to be dispelled from the reservoir 332 and chamber 333, and delivered to the ultimate target “T” (i.e., patient) through a needle 314, as described above with respect to the first exemplary embodiment. The BFS vial 330 may be releasably coupled to injection assembly 310 (e.g., comprising a connection collar 318), which fluidically connects the needle hub 316, and the needle 314 (e.g., selectively covered by a cap 312) to an outlet end 331 of the BFS vial 330, to form the BFS injection system 300, as also described above. The assisted collar 400 can be secured, supported mounted on and/or coupled to the BFS vial, for example, by sliding on or securing over the proximal end 330p of the vial (e.g., over a labeling portion 338 thereof), as illustrated in FIGS. 5C, 5B, which depicts a first exemplary vial 330 and a second exemplary vial 330b as described hereinabove. In some embodiments, the dispensing collar 400 is releasably secured to the BFS vials 330, 330b, and may be designed to be removed therefrom after use and re-used with another BFS vial.

With reference to FIGS. 5A-5E, in the exemplary embodiment the first and second arms 402, 404 are curved inwardly from the pivot point toward a central longitudinal axis of BFS vial 330 in the portion of the arms 402, 404 that are adjacent the vial when mounted thereto. The first and second arms 402, 404 then curve outwardly from the longitudinal axis of the vial 330 and downward as they extend past the fluid reservoir 332 toward the gripping ends 402a, 404a, creating a generally “S” shaped arm. The inward curvatures 416 and 418 that are formed by curving the arms 402, 404 are protrusions that contact and compress the fluid reservoir to increase the force applied by the user and expel the fluid therefrom as described hereinabove. However, in some embodiments, other sizes, shapes, numbers or arrangements of arms 402, 404 are also possible. A support structure 415a, 415b may also be provided on the outer curved surfaces 417a, 417b, in order to increase the stiffness of arms 402, 404 respectively, to facilitate applying the compressive force to the vial 330 during use.

Arms 402, 404, attachment member 406, support structures 415a, 415b, and pivot portions 412, 414 can be made as a single, unitary structure as illustrated, or may be made as separate components joined together. A grip portion 432 and 434 may be provided on an outer surface of the first arm 402 and the second arm 404, respectively to enhance gripping by a user, as described above. A user of the BFS assisted dispensing system 320 can grasp the grip portions 432, 434 (e.g., using a thumb and index finger) to provide the desired compressive force for actuation of the first and second arms 402, 404.

In the illustrated example, attachment member 406 has an opening 410 that is sized and shaped to substantially mate with the proximal end 330p of the BFS vial 330. As such, the size and shape of the opening 410 is determined by the particular BFS vial. As shown in FIG. 6C, opening 410 may be approximately centrally located on attachment member 406, and include notches 409 for receiving a corresponding ridge 313 of the BFS vial 330, should there be one. Other sized and shaped notches may be utilized, depending upon the particular vial. Extending inwardly from either side of the bottom of attachment member 406 are flanges 419a, 419b. The flanges 419a, 419b mate with corresponding indents 330, 332, disposed between the proximal end 330p and the fluid reservoir 332 to support the assisted collar on the exemplary BFS vial 330. Extending upwardly and outwardly from the flanges 419a, 419b on outside of the attachment member 406 are spreading projections 411a, 411b.

Spreading projections 411a, 411b are provided to assist in the insertion and removal of assistive dispensing collar 400 in order to spread flanges 419a, 419b apart a sufficient width to receive the proximal end 330p of the BFS vial 330 therethrough. Projections 411a, 411b may include an exterior grip portion 407a, 407b that may be knurled or other structural features designed to enhance gripping by a user. Upon insertion and removal of the assistive dispensing collar 400 the spreading projections 411a, 411b are grasped by a user and compressed toward each other so that the flanges 419a, 419b are spread apart to allow the proximal end of BFS vial 330 to pass through. The flanges 419a, 419b and spreading projections 411a, 411b may be made of a material that deflects so that once the spreading projections are released, the flanges can return back toward to their original position and support the assisted dispensing collar 400 on BFS vial 330 as shown in the Figures.

Once connected toward the proximal end of the BFS vial 330, the assisted dispensing collar 400 is ready for use. By connecting the dispensing collar 400 as shown in the present exemplary embodiment, the arms 402, 404 provide a point of leverage for a user that is at the distal end of the BFS assistive dispensing system 320, adjacent the injection or distal end. The assisted dispensing collar 400 acts as a fulcrum when the user grips the arms 402, 404 and applies pressure to squeeze the arms 402, 404 toward each other. In this manner, the dispensing collar 400 assists in the manual actuation of the BFS injection system to dispense and/or inject contents from the fluid reservoir 332 of the BFS vial 330 by amplifying or increasing the pressure produced by the user.

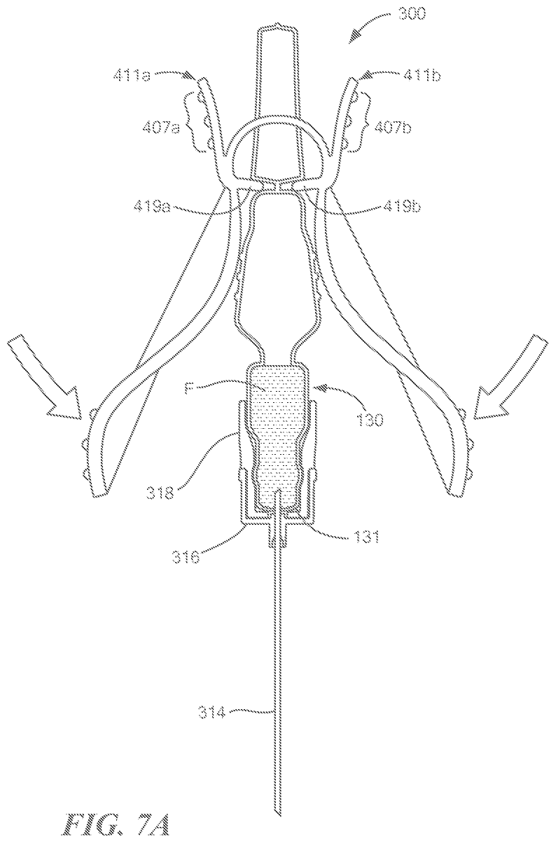

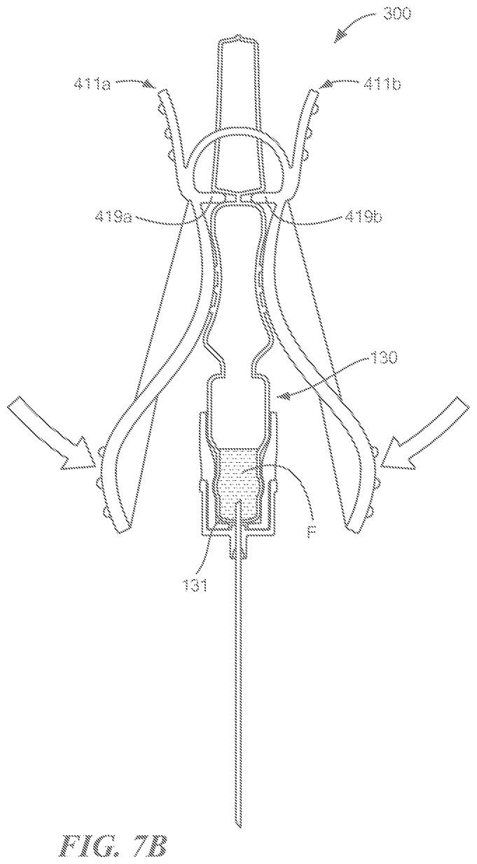

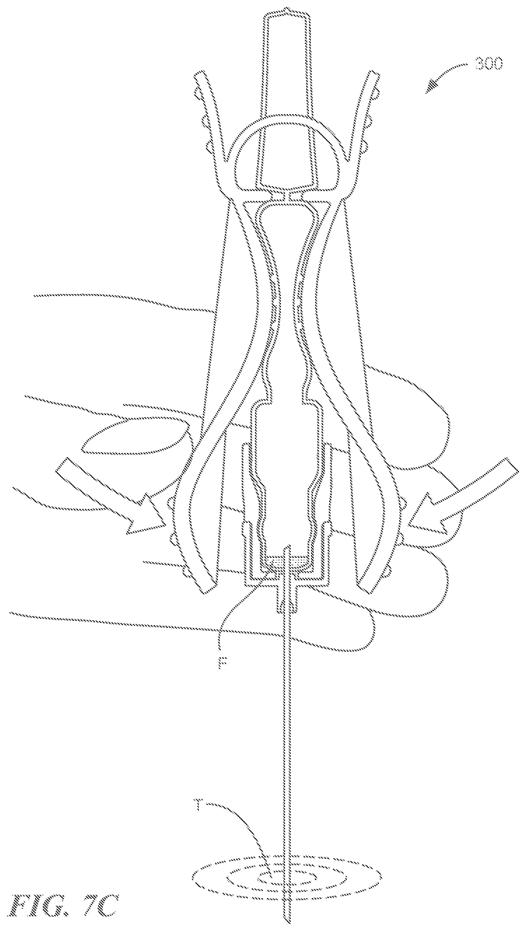

Referring now to FIGS. 7A-7C, once inserted onto the BFS vial 330, the inward curvatures 416 and 418 of first and second arms 402, 404 are located adjacent to the fluid reservoir 332 of the BFS vial 330, for example, in contact therewith (FIG. 7A), without applying a compressive force thereto. This is the first, attached position. Further movement of the first and second arms 402, 404 toward each other, as shown by the arrows of FIG. 7B, causes the inward curvatures 416, 418 to push on the external walls of the fluid reservoir 332/333, thereby applying a compressive force that deforms the fluid reservoir to cause expulsion (e.g., all or some) of the air or other fluid from the reservoir. This is the second, compressed position. Continued pressure applied by the user causes further movement of the inward curvatures 416, 418 toward each other as shown in FIG. 7C into the third, injecting position. The continued pressure applied by the user through the assisted collar 400 forces the air out of the fluid reservoir 332, and into and/or through dispensing chamber 333, and the liquid from the dispensing chamber 333 through the needle 314 and into the target injection site. This occurs without the user having to apply the compressive force that would be needed absent the assisted collar, by virtue of the leverage provided through the assisted dispensing collar 400. Once injected, the assistive collar 400 may be removed and re-used and the empty vial 330 and injection assembly 310 discarded.

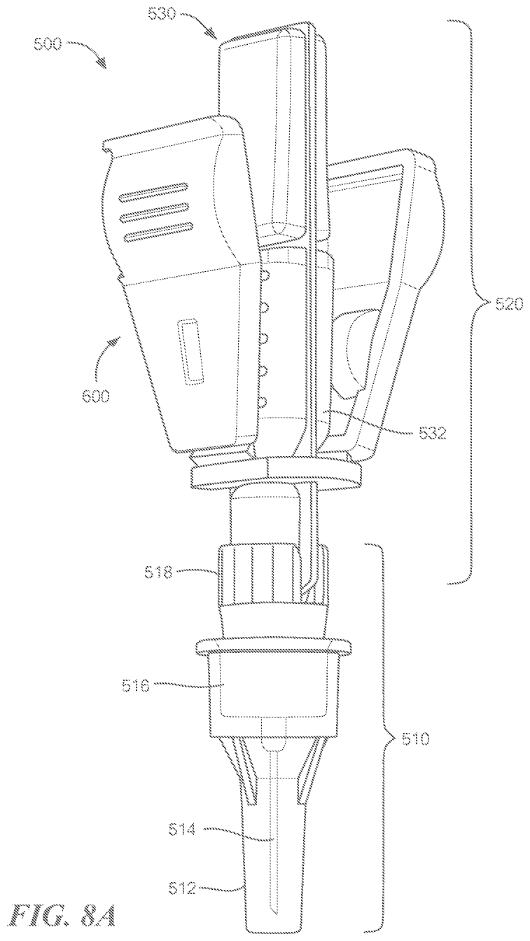

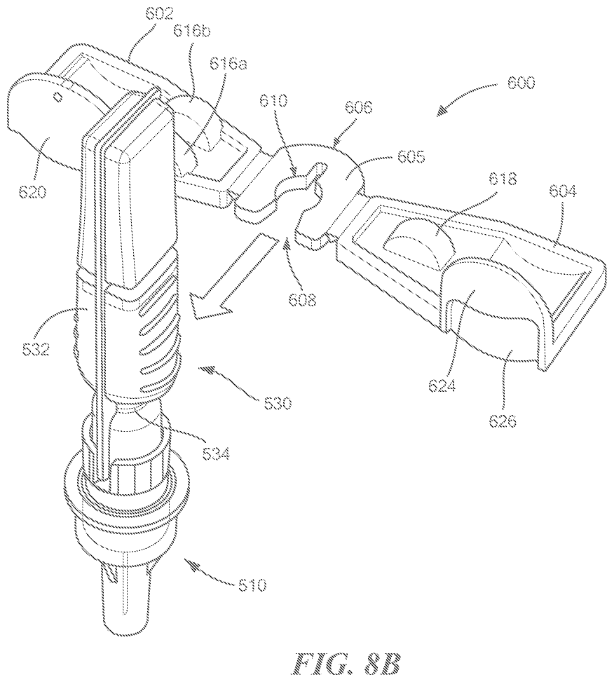

Referring now to FIGS. 8A-10G, a third exemplary embodiment of a BFS assisted dispensing system 520 that employs an assisted dosing and dispensing device, for example a lever and/or collar 600 for actuating at least one fluid reservoir 532 of a BFS vial 530 is illustrated. The assistive dispensing and dosing collar 600 is similar in function to the assistive dispensing collars 200, 400 but the first and second arms 602, 604 are movable between a flat, installation configuration and a folded usage position. In addition, first arm 602 includes a first dosage setting member 620 (also referred to as a first disk), and the second arm 604 includes a second dosage setting member 624 (also referred to as a second disk) that can additionally regulate dosage from the BFS vial 530. For example, the first and second dosage setting members 620, 624 can engage with each other to limit the travel of the first and second arms 602, 604 toward each other, which in turn can limit the amount of compression force the arm protrusions 616a, 616b, 618 can apply to the fluid reservoir 532 and thereby define the amount of fluid expelled therefrom.

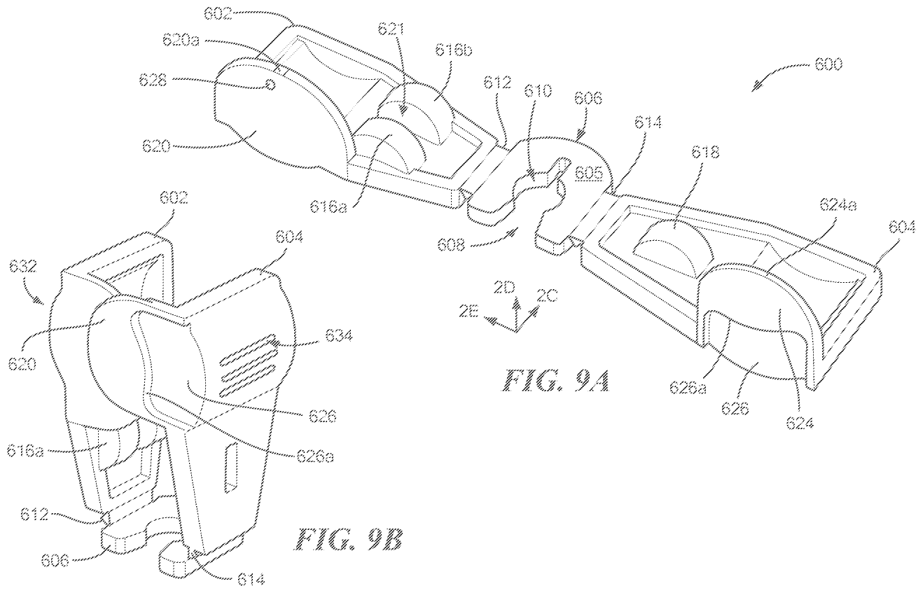

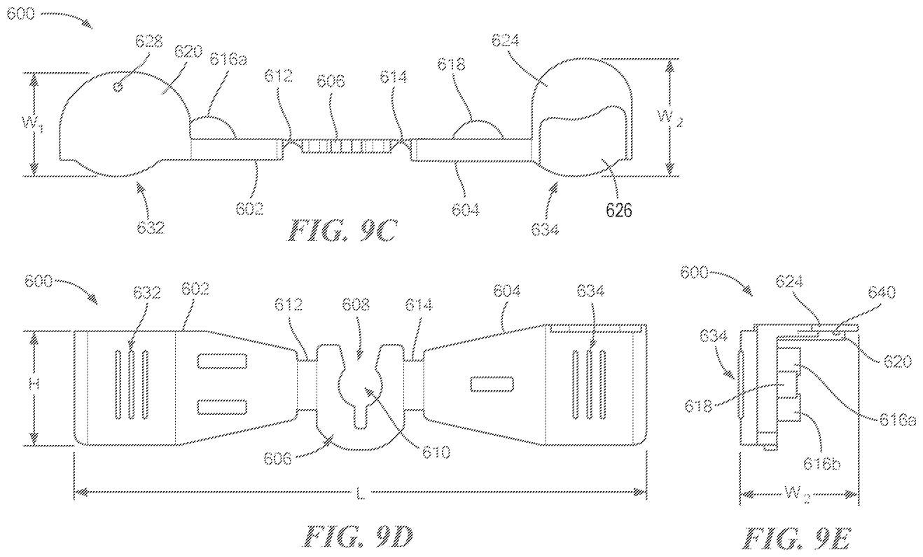

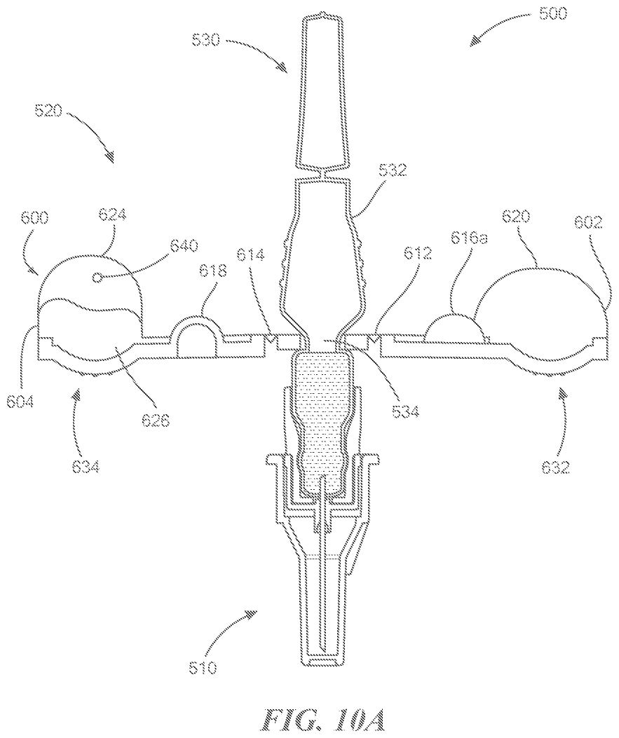

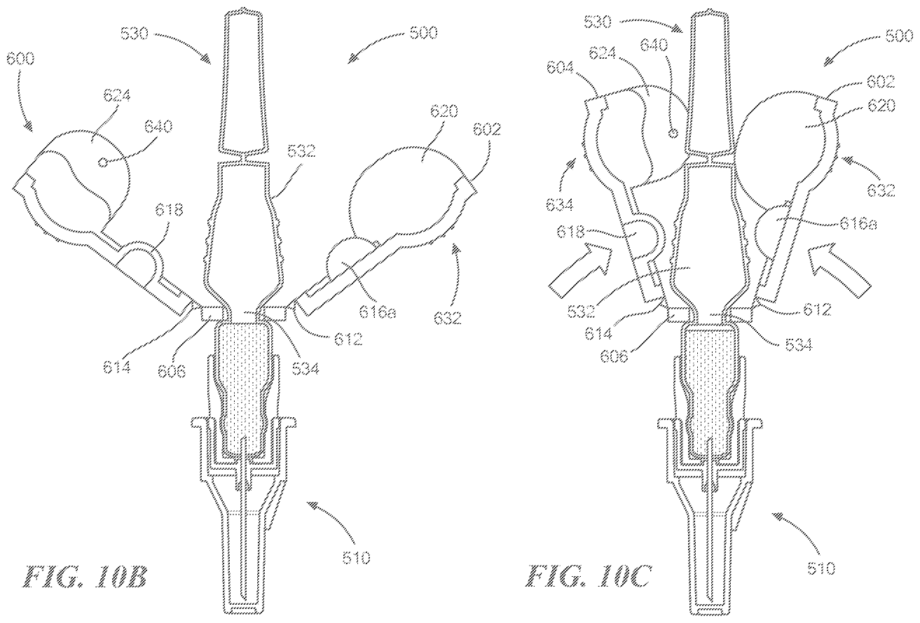

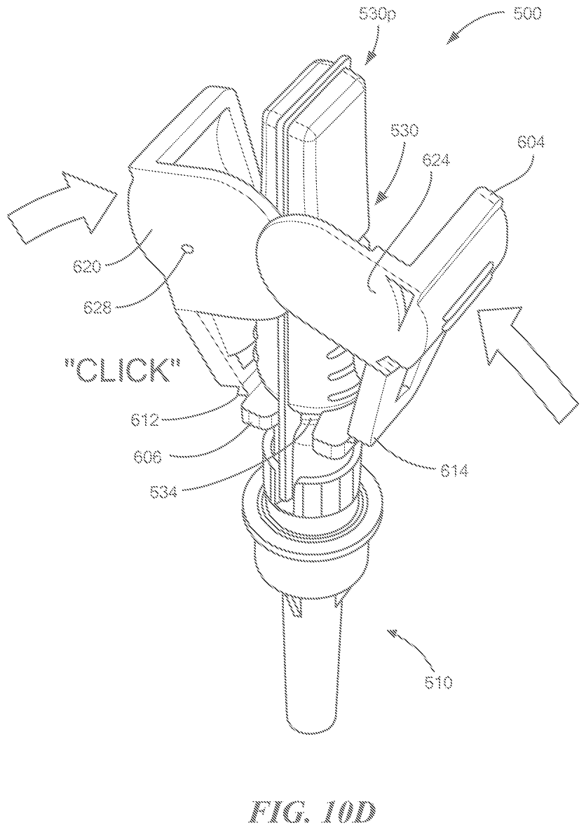

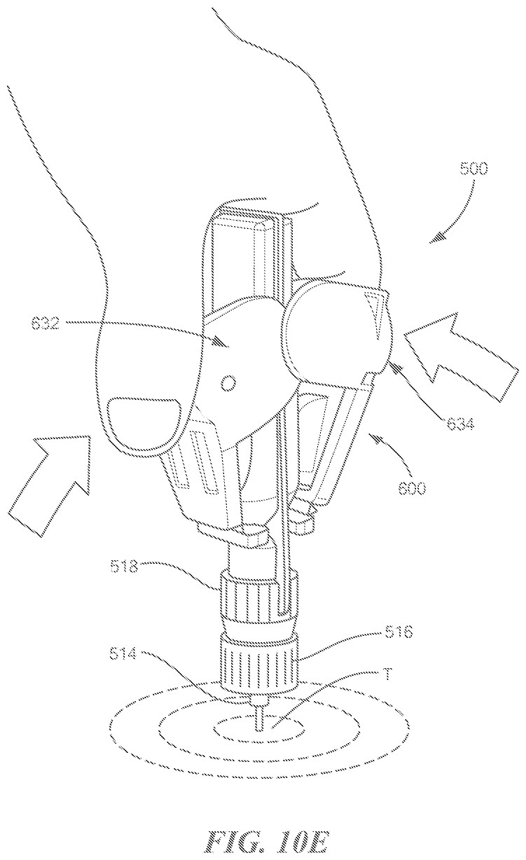

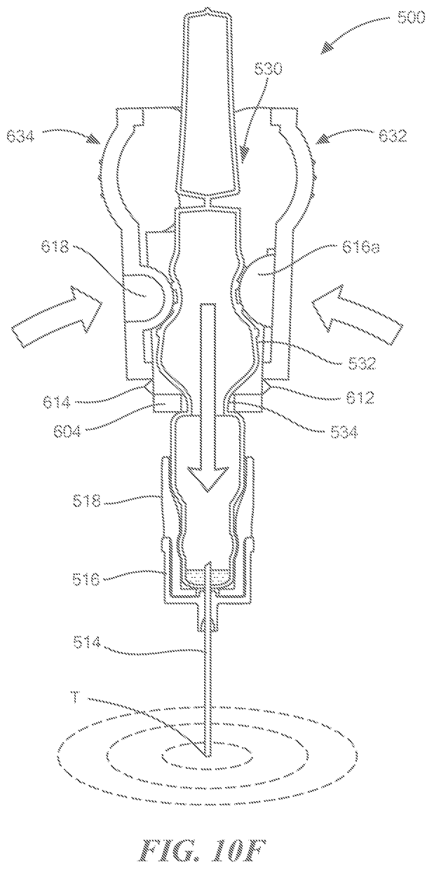

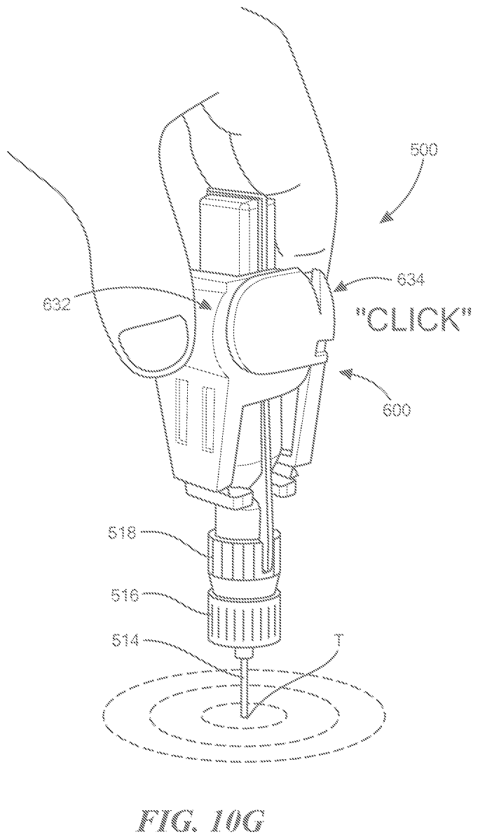

Referring now to FIGS. 9A-9E, the exemplary dispensing collar 600 including first arm 602 and second arm 604 pivotally attached to a support member 606 disposed therebetween, is illustrated. The central support member 606 may include a body 605 made of a flexible material and include an access channel 608 leading to a connector slot 610 that is sized and shaped to mate with a connection portion of the BFS vial 530, as described above with reference to the first exemplary embodiment. The first and second arms 602, 604 can be rotated, i.e. pivoted out of plane with the support member 606 at a first pivot portion 612 and second pivot portion 614, respectively and toward each other for actuating a BFS vial 530 (FIGS. 10A-10F). Thus, the dosing and dispensing collar 600 can be folded from the flat configuration of FIGS. 8B, 10A (e.g., used for installation to the BFS vial) also referred to as the first position, into the folded configurations of FIGS. 10B, 10C where the protrusions can be adjacent to and/or in contact with BFS vial 530 but without applying a compressive force, (also referred to as the folding configurations) to an engaged or injection configuration of FIG. 10D where compressive force is applied to the vial 530 (e.g., the arms 604 are urged radially inward toward a proximal end 530p of the BFS vial 530). First dosage setting member 620 may extend from an outside edge of arm 602 and second dosage setting member 624 may extend from an outside edge of arm 604. As shown in FIGS. 9A-9E the second dosage setting member 624 can have a leading edge 624a that passes over the outer surface of the first dosage setting member 620, such that the first and second dosage setting members have a layered arrangement in side view (FIG. 9B). Travel of the first and second arms 602, 604 toward each other is allowed until leading edge 620a of the first dosage setting member 624 abuts and edge 626a defined by stepped portion 626, thereby defining a final dispensing configuration for the dosing collar 600 that corresponds to a desired dosage.

In the illustrated example, the first arm 602 may also include a pair of inwardly extending protrusions 616a, 616b separated from each other by a gap 621, and the second arm 604 may likewise include a single inwardly extending protrusion 618 disposed at a location corresponding to gap 621. These protrusions are similar to the protrusion previously disclosed in function, but with different numbers, sizes and configurations of the protrusions. However, movement of first and second arms 602, 604 toward each other likewise causes the protrusions 616a, 616b, 618 to push on the external walls of the fluid reservoir 532 (and/or a constriction 534 in communication therewith), thereby applying a compressive force that deforms the fluid reservoir to cause expulsion of fluid from the BFS vial 530 (e.g., via an administration assembly 510 comprising a needle hub 516, a connecting collar 518, a needle 514, and/or a cap 512). Grip portions 632, 634 may be provided as well to aid a user with gripping the assistive dosing and dispensing collar 600 (e.g., using a thumb and index finger) to provide the desired compressive force for actuation of the BFS vial 530, as described hereinabove.

In some embodiments, the dosing collar 600 can have one or more structural features that provide audible feedback, tactile feedback, visual feedback, or any combination thereof when the initial folded configuration is achieved. For example, the second dosage setting member 624 can have a projection 640 (also referred to as a nub) that slides along the upper surface of the first dosage setting member 620 until it is releasably received in a recess 628 or opening in the first dosage setting member, thereby generating an audible and tactile click. Alternatively or additionally, in some embodiments, the dosing collar can have one or more structural features that provide audible feedback, tactile feedback, visual feedback, or any combination thereof when the final dispensing configuration is achieved. For example, the projection 640 of the second dosage setting member 624 can engage with another recess or opening in the first dosage setting member 620, the other recess being disposed farther from the leading edge 620a and at a location that corresponds with a desired compression of the fluid reservoir 532 by projections 616a, 616b, 618 to effect, i.e. regulate dispensing of a desired amount of fluid (e.g., dosage).

In some embodiments, the sizes of components of the dosing and dispensing collar 600, for example, the length of the first and second arms, the size of protrusions 616a, 616b, 618, and/or the sizes of dosage setting members 620, 624 can determine the minimum distance between the protrusions and thereby amount of compression applied to the BFS vial 530. Different dosing collars can thus be used with the same size vial 530 to achieve different desired doses, for example, to use dosing and dispensing collar 600 with a first vial (e.g., having a fluid content of 0.6 ml) to achieve an adult-sized dosing (e.g., all or almost all of the 0.6 ml) and to use child-size dosing collar with a second vial substantially identical to the first vial to achieve a child-sized dosing (e.g., half-dose, or about 0.3 ml). Except for the sizing of dosage setting members, the various components of child-size dosing collar (e.g., arms; protrusions; dosing setting member; recess; leading edges, etc.) and operations thereof can otherwise be substantially identical to adult dosing collar 600. For example, the dosing collar 600 can have a width, W1, at the first dosage setting member 620 of about 15.7 mm (0.62″), a width, W2, at the second dosage setting member 624 of about 17.7 mm (0.69″), a height, H, of about 17 mm (0.67″), and a length, L, of about 85.1 mm (3.35″). For example, the child-sized dosing collar 600 can have a width, W3, at the first dosage setting member of about 17.7 mm (0.69″), a width, W4, at the second dosage setting member of about 17.7 mm (0.69″), a height, H2, of about 17 mm (0.67″), and a length, L2, of about 85.1 mm (3.35″).

In use, the BFS vial 330 is received into place within the slot 610, as shown in FIGS. 8B, 10A. In some embodiments, arms 602, 604 can be rotated about their respective pivot portions 612, 614, out of plane with the support member 606 and toward a central axis of the BFS vial 333, as shown in FIGS. 10B-10C. The rotation of the arms 602, 604 can continue until the dosage setting members 620, 624 engage with other and the dosing collar 600 is in a folded configuration, as shown in FIGS. 10B, 10C. For example, the folded configuration can be where the protrusions 616a, 616b, 618 are in contact with, or at least adjacent to, the exterior of the fluid reservoir 332, but without applying a substantial compressive force thereto. In some embodiments, the dosing collar 600 can provide an audible or tactile click once the initial folded configuration is reached.

With the dosing collar in the folded configuration, the needle cap 512 can then be removed in preparation for injection and a user can hold the BFS injection system 500 by gripping the grip portions 632, 634 of the dosing collar 600. Once positioned with respect to a target “T” (e.g., an exposed surface of a patient), the needle 514 can be advanced into the target by moving the BFS injection system 500 via the dosing collar 600, as shown in FIG. 10E. With the needle 514 inserted into the target T, a dosage of fluid contained in BFS vial 530 can be administered by applying a compressive force between arms 602, 604 of the dosing collar 600, for example, by squeezing grip portions 632, 634 together, as shown in FIGS. 10E-10G. As described above, interaction between the dosage setting members 620, 624 can limit the amount of squeezing (e.g., the minimum distance between the arms), thereby defining a maximum amount of fluid that can be dispensed from the BFS vial 530. In some embodiments, the dosing collar 600 can provide an audible or tactile click once the squeezing limit is reached (10G).

In such a manner, for example, the dosing collars described herein can advantageously be utilized to easily dispense the contents of the fluid reservoir 132 through the needle 114 and into a target, without a user being required to directly squeeze or touch the fluid reservoir 132. As the dosing collars described herein necessarily define larger dimensions and contact surfaces than the fluid reservoir 132 (for it is configured to fit around the fluid reservoir 132), the dosing collar provides increased surface area for which the user to engage. Moreover, the dosing collars can act as a lever that increases the compression force applied to the fluid reservoir 132 by the user, thereby allowing the user (e.g., an arthritic user or other user or reduced physical capabilities) to apply less force to actuate the BFS vial. In some embodiments, the travel limitations imposed by the dosing collars can precisely control the force applied to the fluid reservoir and thereby reliably control the amount of fluid dispensed by the BFS injection system, and the releasable coupling can allow for the dosing collars to be reused with other BFS vials. Other advantages will be readily apparent to one of ordinary skill in the art based on the foregoing description.

In any of the above exemplary embodiments, the needle 114, 314, 514 can be held in place within the target “T” while the compression force is maintained for a predetermined period of time (e.g., 5-10 seconds), after which the needle can be withdrawn from the target. Alternately or additionally, the compression force can be maintained until the needle is completely withdrawn from the target. The BFS injection systems 100, 300, 500 may then be discarded. Alternatively, the assisted and/or dosing collar 200, 400, 600 can be removed from the expended vial 130, 330, 530 for reuse with a new vial, and the injection assembly 110, 310, 510 and the expended vial 130, 330, 530 attached thereto can be discarded.

### III. Rules of Interpretation

Throughout the description herein and unless otherwise specified, the following terms may include and/or encompass the example meanings provided. These terms and illustrative example meanings are provided to clarify the language selected to describe embodiments both in the specification and in the appended claims, and accordingly, are not intended to be generally limiting. While not generally limiting and while not limiting for all described embodiments, in some embodiments, the terms are specifically limited to the example definitions and/or examples provided. Other terms are defined throughout the present description.

Neither the Title (set forth at the beginning of the first page of this patent application) nor the Abstract (set forth at the end of this patent application) is to be taken as limiting in any way as the scope of the disclosed invention(s). Headings of sections provided in this patent application are for convenience only, and are not to be taken as limiting the disclosure in any way.

All definitions, as defined and used herein, should be understood to control over dictionary definitions, definitions in documents incorporated by reference, and/or ordinary meanings of the defined terms. The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

The indefinite articles “a” and “an,” as used herein in the specification and in the claims, unless clearly indicated to the contrary, should be understood to mean “at least one” or “one or more”.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise. For example, the enumerated list “a computer, a laptop, a PDA” does not imply that any or all of the three items of that list are mutually exclusive and does not imply that any or all of the three items of that list are comprehensive of any category.

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention(s) may be practiced with various modifications and alterations, such as structural, logical, software, and electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

Devices that are in communication with each other need not be in continuous communication with each other, unless expressly specified otherwise. On the contrary, such devices need only transmit to each other as necessary or desirable, and may actually refrain from exchanging data most of the time. For example, a machine in communication with another machine via the Internet may not transmit data to the other machine for weeks at a time. In addition, devices that are in communication with each other may communicate directly or indirectly through one or more intermediaries.

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

It will be understood that various modifications can be made to the embodiments of the present disclosure herein without departing from the scope thereof. Therefore, the above description should not be construed as limiting the disclosure, but merely as embodiments thereof. Those skilled in the art will envision other modifications within the scope of the invention as defined by the claims appended hereto.

What is claimed is:

1. An accessory system for a medical dispensing device, comprising:
an assistive dispensing collar comprising an attachment member including at least one opening sized to mate with a portion of a blow-fill-seal (BFS) vial defining a fluid reservoir, a first arm and a second arm pivotally connected on opposing sides of the attachment member, each of the first and second arms including a gripping end and a pivoting end, and at least one protrusion;
wherein upon a mating of the at least one opening of the assistive dispensing collar with the portion of the BFS vial, the at least one protrusion of each of the first and second arms are disposed in a first position adjacent the fluid reservoir, and
wherein in response to receiving a first compression force exerted at the gripping end of the assistive dispensing collar, the first and second arms transition to a second position where the at least one protrusion of each of the first and second arms apply a corresponding second compression force to the fluid reservoir so as to cause contents of the fluid reservoir to be dispensed.

2. The accessory system of claim 1, wherein the attachment member mates with a connection portion of the BFS vial disposed adjacent a proximal end of the vial and acts a lever for generating the second compression force.

3. The accessory system of claim 2, wherein the at least one protrusion comprises at least one inward curvature formed by the body of the first and second arms.

4. The accessory system of claim 3, wherein the first and second arms comprise a generally S shape curvature and wherein each of the first arm and second arm include a support structure supported on an outer curved surface of each of the first arm and second arm to increase the stiffness of each arm to facilitate application of the second compressive force.

5. The accessory system of claim 3, wherein the attachment member further comprises a pair of flanges extending inwardly from a portion of the attachment member, the pair of flanges constructed and arranged to support the assistive dispensing collar on the BFS vial.

6. The accessory system of claim 5, further comprising a pair of spreading projections extending from the attachment member and including a gripping surface, wherein in response to receiving a third compression force exerted on the gripping surface the spreading projections move the flanges apart a sufficient width to receive a portion of the vial.

7. The accessory system of claim 1, wherein the attachment member mates with a connection portion of the BFS vial disposed below the fluid reservoir and acts a lever for generating the second compression force.

8. The accessory system of claim 1, wherein the at least one protrusion includes at least one arcuate portion.

9. The accessory system of claim 8, wherein the at least one arcuate portion includes at least one extension portion.

10. The accessory system of claim 1, wherein the first arm includes a pivoting end adjacent the attachment member and a gripping end opposite the pivoting end.

11. The accessory system of claim 10, wherein the gripping end of the first arm and the second arm is adjacent a proximal end of the BFS dispensing system.

12. The accessory system of claim 9, wherein the gripping end of the first arm and the second arm is adjacent a distal end of the BFS dispensing system.

13. The accessory system of claim 1, wherein the first arm includes a first dosage setting member, the second arm includes a second dosage setting member, and the first and second dosage setting members engage with each other during the application of the first compression force to limit further movement of the one or more protrusions.

14. The accessory system of claim 13, wherein the assistive dispensing collar is constructed to produce audible or tactile feedback when the limit of movement of the one or more protrusions is reached.

15. The accessory system of claim 1, further comprising at least one gripping portion disposed on an outer surface of the assistive dispensing collar.

16. The accessory system of claim 1, further comprising an injection assembly designed to be secured to a distal end of the BFS vial.

17. The accessory system of claim 1, wherein the assistive dispensing collar is removably attached to the vial through the opening in the attachment member.

18. An accessory system, comprising:
an assistive dispensing collar comprising an attachment member including at least one opening sized to removably attach the dispensing collar with a portion of a blow-fill-seal (BFS) vial comprising a collapsible fluid reservoir containing a liquid, a first arm and a second arm pivotally connected on opposing sides of the attachment member, each of the first and second arms including a gripping end and a pivoting end and at least one protrusion designed to apply pressure to the fluid reservoir upon engagement with the fluid reservoir;
wherein the assistive dispensing collar is configured to, in response to receiving a first compression force, move the one or more protrusions from a first attached position to a second compressed position where the one or more protrusions apply a force sufficient to begin compressing the fluid reservoir to dispense the fluid out of the fluid reservoir.

19. The accessory system system of claim 18, wherein the first arm and the second arm are movable between different stages of engagement as compression is increased, with the final stage of engagement dispensing the liquid.

20. The accessory system of claim 18, wherein the second compression force is along a direction substantially perpendicular to a longitudinal axis of the fluid reservoir.
